(19) United States
(12) Patent Application Publication
(10) Pub. No.: US 2002/0090740 A1
(43) Pub. Date: Jul. 11, 2002

(54) Method and apparatus for performing an assay

(76) Inventor: Scott A. Lesley, San Diego, CA (US)

Correspondence Address: TIMOTHY L. SMITH, GENOMICS INSTITUTE OF THE, NOVARTIS RESEARCH FOUNDATION, 10675 JOHN JAY HOPKINS DRIVE, SUITE E225, SAN DIEGO, CA, 92121-1127, US

(21) Appl. No.: 10/011,374

(22) Filed: Sep. 25, 2001

Related U.S. Application Data
(63) Continuation-in-part of application No. PCT/US01/19971, filed on Jun. 22, 2001.
(63) Continuation-in-part of application No. 09/602,245, filed on Jun. 23, 2000.

(51) Int. Cl. G01N 33/53; G01N 33/542; G01N 33/543

(52) U.S. Cl. 436/518; 435/7.9

(57) ABSTRACT

This invention provides apparatus and methods that enable the direct measurement of an analyte sample in an assay well by forming an independent measuring region. The sample to be measured can be separated into portions, for example, a bound portion and a free portion. To form the independent measuring region, a layer-forming material that can restrict the passage of radiant energy that is measured by a detection device is added to the assay well. The layer-forming material forms a layer that restricts the passage of radiant energy from the region. Thus, a sample portion in one separate region may be measured by a detection device separately and independently of a portion of the sample in a different region. In one example, a second portion in another region can be separately and independently measured by a detection device without the removal of the portions from the assay well.

### CROSS-REFERENCES TO RELATED APPLICATIONS

This application is a continuation-in-part of International Application No. PCT/US01/19971, filed Jun. 22, 2001, which application is a continuation-in-part of U.S. Ser. No. 09/602,245, filed Jun. 23, 2000. These applications are incorporated herein by reference for all purposes.

### BACKGROUND OF THE INVENTION

1. Field of the Invention

This invention pertains to the field of biotechnology, and in particular, to methods and an apparatus for conducting assay measurements. The method and apparatus are more particularly useful in performing assays for the purpose of detecting bound and free analyte.

2. Background

An assay system is generally utilized to evaluate the interaction between components in a confined environment. These assay systems are used in a wide variety of chemical, biological, and medical disciplines, as well as having both commercial and research applications. As such, assay systems need to be rapid, specific, and sensitive. Assay systems may be more specifically identified according to particular measurement techniques. For example, an immunoassay investigates and measures antigen-antibody interactions. Immunoassays are particularly useful because of the their high level of specificity and sensitivity. In conjunction with high throughput screening processes, hundreds of thousands of immunoassays may be analyzed each day.

Another type of assay is the radioimmunoassay, which explores and measures antigen-antibody interactions by monitoring a radioisotopically labeled (“radiolabeled”) antigen. Because of the use of radioisotopes, radioimmunoassays provide very sensitive and specific assays on an atomic or molecular level. As another example, enzymatic assays monitor enzyme-substrate interactions. Enzymatic assays are also very specific and are often detected through calorimetric analysis. Enzymes are often conjugated to antibodies as a convenient means of measuring the amount of antibody present via the enzymatic activity.

Unfortunately, current measurement techniques for each of the assays identified above involve intricate, often time consuming steps, such as centrifugation and washing steps that hinder automated processes. Frequently, assay measurement requires physical removal of the analyte from the sample container in order to distinguish different analyte components. These additional processing steps decrease the accuracy of the measurement as well as increase the cost. In addition, removal of the analyte and subsequent washing steps make it difficult to measure low affinity interactions with fast off rates.

The assays described above typically depend on measuring the concentration of a bound analyte relative to a free, or unbound, analyte. The bound analyte is composed of the analyte and a binding agent, usually a molecule that binds to the analyte in a biological system. The free analyte is not bound to the binding agent. For example, immunoassays depend on the binding of an antigen to a specific antibody. The bound analyte is the antigen-antibody complex. The free analyte is the antibody alone. Similarly, an enzymatic assay depends on a substrate binding to a specific enzyme. The binding agent may also be a detective marker that allows the measuring device to quantify the amount of bound analyte to determine the level of the detective marker. In a radioimmunoassay, the analyte is a radioisotopic marker. Frequently, the goal is to evaluate the affinity of one molecule for another. This affinity may be quantified by separating the bound analyte of the sample from the unbound analyte into two portions.

Current methods of measuring the ratio of bound to unbound analyte generally separate the bound and unbound analyte into two portions and measure only one of the portions. Accordingly, the method only infers the measurement of the other portion by calculating the difference between the total amount of analyte and the amount of analyte measured in the first portion. Unfortunately, separation requires at least one and usually more than one extra manipulation step. For example, separation by centrifugation involves the initial centrifugation step, separation of the two portions into two separate containers, followed by repeated washing of the bound portion. The extra steps increase the time of the experiment and may cause physical loss of the material to be measured, resulting in an inaccurate measurement. Further disadvantages include the increased cost of adding extra separation steps to the process and the cost of the washing liquid.

Separation steps may also disturb the thermodynamic equilibrium between the bound and free steps due to the extra washing with solution, which allows ligands with rapid dissociation rates to be released during the wash procedure. In order to accurately measure the affinity of the binding interaction, it is necessary to measure the proportion of bound and free analyte in the same sample simultaneously.

Another disadvantage of the conventional methods currently available is the inability to measure samples in situ, i.e., directly in the assay well without any interference or manipulation of the designed environment. For example, radioimmunoassay procedures require a separation step because the measuring device cannot distinguish the radiation bound to the analyte from the radiation free from the analyte in situ. The radiolabeled portion must first be separated and then measured. Similarly, enzyme assays require the bound protein enzyme complex to react with a colorimetric agent. In order to directly measured the bound enzyme, it must first be separated from the unbound portion and calorimetric agent. This also requires a separate step that is not only time consuming, but also results in the loss of analyte, thus, introducing errors into the analysis. Also, fluoroimmunoassays utilize fluorescing agents as detective markers. Typically, the unbound fluorescing agent must be separated from the bound portion prior to quantitation.

In all of these known, conventional methods, the amount of bound and free analyte is quantified by directly measuring the bound analyte and inferring the amount of free analyte by calculating the difference between the starting amount of total analyte and subtracting the amount of bound analyte measured. Allowances for analyte loss during the separation and handling steps are estimated, resulting in increased margins of error and decreased accuracy in measurements.

In an attempt to overcome some of these disadvantages, U.S. Pat. No. 5,017,473 provides for a chemiluminescent immunoassay without removing the bound and free analyte portions from the sample container. However, detection of the unbound sample is accomplished by indirectly measuring a reference standard under exact conditions, thereby suffering from similar inaccuracies as described above. Moreover, in this invention, the bound portion must be attached to a solid support, thus perhaps interfering with the conformation and binding properties of the binding molecule that would otherwise be free in solution. Further, attachment to a solid support does not exactly mimic the in vivo biological environment where, for example, the enzyme, cell, or antigen is floating free in solution.

Conventional technology generally requires that assays measure only one component directly, while another component must be indirectly calculated. Also, the conventional methods necessarily employ extraneous manipulation steps that add time and cost to the experiment. Thus, there exists a need for a cost effective, time saving assay technique that more accurately measures analyte interactions as they exist in present or proposed biological systems. The present invention fulfills these and other needs.

### SUMMARY OF THE INVENTION

The present invention provides a cost effective, time saving, accurate assay measurement system that is readily automated. In some embodiments, the invention provides an assay that separates a sample into a directly and independently measurable region. The invention provides a directly measurable region in situ, thus obviating the need for extra washing and centrifugation steps. To overcome the disadvantages in conventional assay systems, herein is provided a method and apparatus for performing an assay measurement by directly measuring an independent assay region.

Briefly, the apparatus and method of the present invention enables the direct measurement of an analyte sample in an assay well by forming an independent measuring region. The sample to be measured may be separated into portions, for example, a bound portion and a free portion. To form the independent region, a layer-forming material is selected to restrict the passage of radiant energy that is measured by a detection device. The selected layer-forming material is added to the assay well and forms a layer that restricts the passage of radiant energy from the region. Thus, a portion in one separate region may be separately and independently measured by a detection device. In one example, a second portion in another region may be separately and independently measured by a detection device without the removal of the portions from the assay well.

In some embodiments, the invention provides a method of performing an assay. These methods involve providing a sample that comprises a detectable label, wherein the sample is present in an assay vessel and at least a portion of the detectable label becomes associated with a surface of assay vessel; adding to the sample a layer-forming material that forms a barrier between detectable label that is associated with the surface of the vessel and, if present, detectable label that is not associated the surface of the vessel; and determining the amount of detectable label that is associated with the surface and/or that is not associated with the surface. The methods can involve determining both bound and unbound detectable label. The detectable label in some embodiments is a radiolabel and the amount of detectable label determined by adding to the sample a liquid scintillent and measuring the luminescence resulting from the interaction of the radiolabel and the scintillent.

It is an advantage of the present invention that the inventive assay system obviates the cost of extra washing and separating steps because the layer which is formed directly in the assay well effectively separates the analyte portions, for example, separating bound and free analyte. Since the formed region is separated by the layer inside the assay well, the costs incurred by using extra steps, such as centrifuging, physical removal of one of the portions from the assay well, and washing any residual remaining in the assay well, may be eliminated or reduced. Additionally, by eliminating these extra steps, the present invention saves time over the current technology.

It is a further advantage of the present invention that the apparatus and method results in more accurate measurements over the conventional, known systems. Because the layer creates a separately detectable region within the assay well, the separate region may be measured directly and independently without removing a portion from the sample well. For example, the separate and distinct region may hold bound analyte for measurement. In one example, another separate region is created that holds the unbound analyte. Accordingly, the amount of both bound analyte and free analyte may be measured directly inside the assay well. Such direct, independent measurement is more accurate and eliminates the need to estimate the amount of free analyte by inferring this amount from the difference between the total analyte added to the assay well and the amount measured in the bound portion, or vice versa. By measuring the bound or free analyte directly and independently, the margin of error is reduced. Also, measuring the portion of analyte as described prevents the loss of analyte that invariably occurs during removal of one of the portions, also resulting in more accurate measurements.

Yet another advantage of the present invention lies in its ability to measure the analyte sample without disrupting the equilibrium within the assay well. The present invention generally avoids the extra washing and removal steps of the conventional methods, thereby more closely mimicking biological systems in vivo. For example, many assays are designed to mimic existing or proposed biological systems. The process of removing one portion, such as the free or bound analyte from the assay well, as well as imposing extra washing steps disrupts the mimicked environments. Accordingly, by eliminating those steps, the present invention more accurately measures environments designed to mimic existing or proposed biological systems.

These and other features and advantages of the present invention will be appreciated from review of the following detailed description of the invention, along with the accompanying figures in which like reference numerals refer to like parts throughout.

### BRIEF DESCRIPTION OF THE DRAWINGS

FIG. 1 is a flowchart diagram of an assay system in accordance with the present invention;

FIG. 2 is a partial cross sectional view of an assay well containing analyte and a layer;

FIG. 3 is a diagram of the assay apparatus; and

FIG. 4 is a partial cross sectional view of a detection device used in conjunction with the present invention.

It should be understood that the drawings are not necessarily to scale and that the embodiments are sometimes illustrated by graphic symbols, phantom lines, diagrammatic representations and fragmentary views. In certain instances, details that are not necessary for understanding the invention or which make other details difficult to perceive may have been omitted. It should be understood, of course, that the invention is not necessarily limited to the particular embodiments illustrated herein.

### DETAILED DESCRIPTION

The invention provides methods and instruments for performing assays and other reactions in a single reaction vessel. The invention allows one to separate a sample into two separate regions, each of which can be separately subjected to different reactions or measurements. Typically, the reactions involve the binding of one or more reagents to a surface, generally the bottom surface, of the reaction vessel. A layer-forming material is then added to the reaction vessel and allowed to form a layer that separates the sample into two separate regions, one on either side of the layer. The separation allows one to, for example, measure the amount of a detectable label that is present in the bottom (bound) region without interference from, for example, unbound detectable label that is present in, usually, the top region. Because no washing or other extraction of unbound components of an assay or other reaction is required, the methods of the invention are particularly suitable for high-throughput screening approaches.

1. Assays

The methods of the invention are suitable for a wide range of assays and other reactions. Basically, the invention is applicable to any assay or reaction in which it is desired to discriminate between a bound and an unbound component. The bound component is generally attached, directly or indirectly, to a surface of the reaction vessel. In some embodiments, for example, the methods of the invention are used to separate detectable label that is bound to a desired target from unbound label that would otherwise interfere with the detection and quantitation of the bound label. Previously, the unbound label would be removed by, for example, one or more centrifugation and/or washing steps. Bound versus free label can be measured simply by making measurements from the top (typically unbound label) and bottom (typically bound analyte) of the reaction vessel.

The assay methods can be applied to many types of binding assays. In some embodiments, for example, a binding moiety that binds to a target analyte can be attached to the bottom of a reaction vessel. The binding moiety and target analyte can be any of a large number of binding pairs. Such binding pairs can be, for example, antibody-antigen, nucleic acid-complementary nucleic acid, carbohydrate-lectin or carbohydrate-antibody, receptor-ligand, etc. A sample that may contain the analyte is then added to the vessel, and the analyte is allowed to bind to the binding moiety. The analyte, if not already labeled, is labeled using a detectable label. Unbound detectable label and bound detectable label are then separated according to the invention.

The separation of bound and unbound components is accomplished, according to the invention, by introducing into the reaction vessel a layer-forming material. The layer-forming material has the physical characteristic of being positionable between the portions in the assay well that are to be separated. For example, the material generally has a higher density than the analyte found in a top portion and a lower density than the analyte found in a bottom portion of the assay well. The material is also capable of forming a layer, for example, by gravity or by low gravity centrifugation. In some embodiments of the invention, the layer-forming material is generally a particulate material of sufficient density to settle to the bottom of the reaction vessel, thus forming a physical boundary between the sample components that are associated with the bottom of the well and those that are not. Alternatively, if, for example, a density gradient exists in the sample, the physical boundary can form at a location other than the bottom of the vessel.

The layer-forming material is conveniently introduced as a suspension of particles in liquid, thus allowing the formation of the boundary layer to be accomplished using liquid handling systems that are generally present in automated screening and reaction systems. It will be appreciated that the material may also be added, for example, as a solid, a gel, or other forms. The material can be added manually or robotically.

For use in the assays of the invention, the layer-forming material is preferably opaque in that it restricts the passage of radiant energy measured by a detecting device that is used to measure the detectable label. It will be appreciated that different assay measurement devices may require a different opaque material to be selected. For example, in a radioimmunoassay, the detecting device may contain a photomultiplier tube and detect radioactive particles. In a radioimmunoassay, regions may be formed where one region contains unbound radioactive particles, and another region contains bound radioactive particles. The opaque material selected for use in the radioimmunoassay, when formed into a layer of sufficient thickness, substantially restricts the passage of radioactive particles between regions. In another example, such as a fluoroimmunoassay, the detecting device may detect fluorescent energy. Consequently, the opaque material selected for use in a fluoroimmunoassay substantially restricts the passage of fluorescent energy through the layer formed between regions.

The opaque material can be, for example, a particulate material, a material chemically or biologically unreactive to the analyte sample, a chromatography resin, a paramagnetic material, or may contain reagents or detection markers that react with the analyte. If the analyte sample contains cells, the opaque material is, in some embodiments, non-toxic to the cells. In a preferred example, the opaque material is paramagnetic silica particles, which restricts the passage of fluorescent energy. Commercially available suitable paramagnetic silica particles include, for example, MagneSil™ (Promega Corporation, Madison Wis.) and Dynabeads™ (Dynal Biotech, Oslo, Norway). Non-magnetic particles that can be used to form a barrier layer are commercially available from, for example, Dynal Biotech.

The use of paramagnetic particles as the sedimentary material is preferred in some embodiments because of the ease with which such particles can be removed from the reaction vessel. A magnetic tip can be introduced into the vessel to remove the particles. The application of a magnetic force to the particles in the reaction vessel can also act as a “magnetic valve” by which one can open and close the barrier between the two separate regions. By opening the “valve,” one can simultaneously add reagents to both the top and bottom of a reaction vessel, after which the magnetic force is released and the barrier layer allowed to re-form.

The detectable labels used in the invention can be primary labels (where the label comprises an element that is detected directly or that produces a directly detectable element) or secondary labels (where the detected label binds to a primary label, as is common in immunological labeling). An introduction to labels, labeling procedures and detection of labels is found in Polak and Van Noorden (1997) Introduction to Immunocytochemistry, 2nd ed., Springer Verlag, N.Y. and in Haugland (1996) Handbook of Fluorescent Probes and Research Chemicals, a combined handbook and catalogue published by Molecular Probes, Inc., Eugene, OR. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocyanate (TRITC), etc.), digoxigenin, biotin, phycoerythrin, AMCA, CyDyeS™, and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectral calorimetric labels such as colloidal gold or colored glass or plastic (e.g., polystyrene, polypropylene, latex, etc.) beads. Semiconductor nanocrystal probes (e.g., Qdot™ nanocrystals, Quantum Dot Corporation, Hayward, Calif.) are well suited for use in the assays of the invention (see, e.g., U.S. Pat. Nos. 5,990,479, 6,207,392, and 6,207,229).

The invention provides, in some embodiments, an alternative to the commonly used scintillation proximity assay. A radiolabeled detectable marker is bound to a target which is bound to the bottom of the reaction vessel. Liquid scintillent is added to the vessel, as is the sedimentary material. Radioactivity associated with the target is detected in the lower phase as a luminescent reaction with the scintillent. Unbound radioactivity can be detected in the upper phase in a similar manner.

The label can be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

The assays are preferably performed in a reaction vessel that allows detection of the detectable label through bottom of the reaction vessel, as well as through the top of the vessel. Clear-bottom microtiter plates are particularly suitable for high-throughput applications of the assays of the invention.

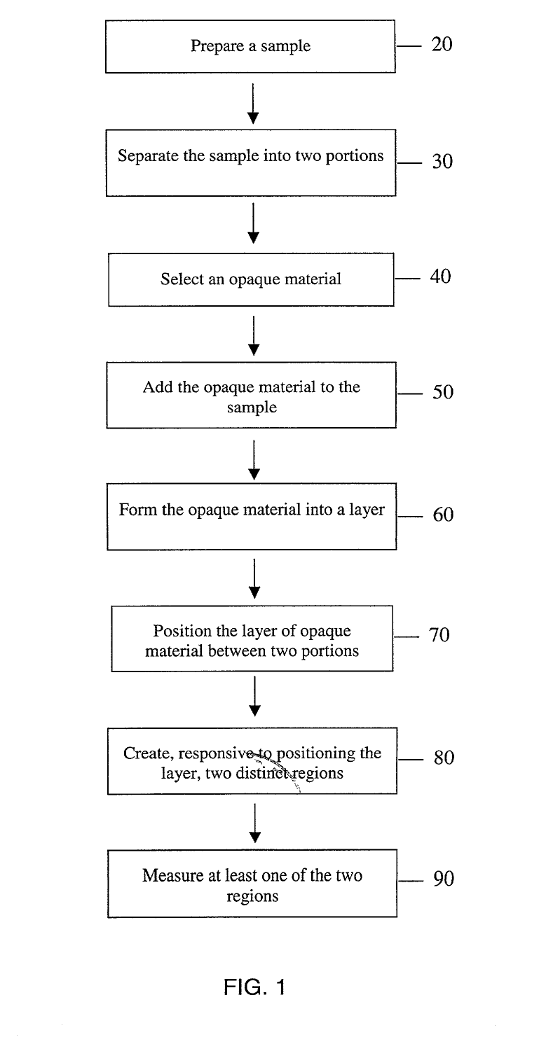

Referring now to FIG. 1, a preferred method of performing an assay on a sample is shown in accordance with the present invention. The method enables the direct and independent measurement of the sample by creating a distinct region which may be separately measured. By directly and independently measuring the region, the assay measurement method is more convenient, efficient and accurate than known, conventional methods of assay measurement. The assay may be, for example, an immunoassay, a fluoroimmunoassay, a radioimmunoassay, or an enzymatic assay. However, it will be appreciated that other assays may be utilized as well. In a particular example of the method, two independent regions may be created.

In block 20, a sample containing analyte to be measured is provided in an assay well. The sample may be, for example, cells expressing a specific antigen. In another example, the sample may be antibody labeled with a fluorescent dye or radioactive marker. As a further example, the sample may contain molecules binding to a ligand, such as an enzyme and its substrate. Of course, it will be appreciated that the sample may be any other sample material suitable for assay measurement. The assay well may be, for example, a microtiter plate, which contains an arrangement of many assay wells, and may be used in a high throughput system. It will be appreciated that the assay well may be arranged in other configurations, such as a row of individual assay wells or a single assay well.

Block 30 shows the analyte sample in the assay well being separated into portions. In one example, gravity separates the analyte into portions. In a particular example, two portions are separated, with the first portion containing substantially all of a bound analyte and the second portion containing substantially all free analyte. In this context, substantially means statistically significant. While gravity may be used to separate the sample into portions it will be appreciated that other techniques, such as centrifugation, or surface adsorption may be used. Of course, it will be appreciated that other methods of separating the analyte into portions may be used.

An opaque material is selected in block 40. In block 50, the opaque material is added to the assay well. Block 60 shows the opaque material being formed into a layer. The layer may be formed by allowing the opaque material to settle by gravity over, for example, bound analyte at the bottom of the assay well. Alternatively, the layer may be formed by applying a low gravity centrifugal force, such as centrifugation. It will be appreciated that the opaque layer may be formed by other methods as well. The layer is of sufficient thickness to prevent measurable passage of radiant energy of the type detected in the assay measurement. In a preferred example, the opaque material is paramagnetic silica particles and the formed layer prevents the passage of fluorescent energy from one portion to another portion.

In block 70, the layer is positioned between portions of analyte. The layer may be positioned, for example, by gravity or centrifugation. In a preferred embodiment, the layer is positioned between a bound portion and a free portion.

In block 80, the formed layer separates the sample into an independently measurable region. In the described example, a second independent region is also created. Preferably, each independent region holds a separate portion for independent measurement. For example, two regions may be created by the layer, with one region containing a bound portion and the other region containing an unbound portion. It will be appreciated that the radiant energy in one region is measurably isolated from the radiant energy in the other region. In another example, the bottom portion may contain cells. The formed layer effectively separates the top portion from the bottom portion such that washing of the top portion does not result in cell erosion of the bottom portion.

Block 90 shows the measurement of radiant energy by a detection device of at least one of the regions created by the opaque layer. It will be appreciated that the radiant energy of either or both regions may be measured directly and independently by one or more detection devices. It will further be appreciated that the radiant energy of the regions may be measured either at the same time or sequentially. Further, the regions may be measured by the same or separate detection device. It will also be appreciated that the measurement of the radiant energy in either or both regions occurs while the analyte is inside the assay well. When measuring the radiant energy in one region, the detection device does not detect the radiant energy contained in the other region.

2. Cell-based assays

The present invention also provides cell-based assay methods. Generally, cells are allowed to become associated with a surface of the reaction vessel. A layer-forming material is added to the reaction vessel and allowed to form a barrier, with the surface-associated cells and substances that are bound to the cells on one side of the barrier, and other unbound reagents on the other side of the barrier. No washing, centrifugation, or other steps are required in order to remove these unbound reagents from the reaction vessel.

Cell-based assays of the invention can use indirect labeling of the cells. For example, one can contact the cells with a labeled antibody, lectin, or other binding agent that binds to a particular cell-surface feature (e.g., a cell-surface protein, carbohydrate, or other structure). The cells are allowed to become associated with a surface of the reaction vessel, generally the bottom surface. The layer-forming material is added to the well and allowed to form a barrier, with the surface-associated cells and labeled binding agent that is bound to the cells on one side of the barrier and the unbound labeled binding agent on the other side of the barrier. Measurements of the detectable label on either or both sides of the barrier are obtained.

Cells also can be directly labeled. For example, the cell can express a reporter gene that, when expressed, produces a readily detectable product. The resulting reporter gene construct is conveniently introduced into cells as part of a “reporter plasmid” or other suitable delivery vehicle (e.g., viral vector, liposome, and the like). A variety of reporter gene systems are known, such as the chloramphenicol acetyltransferase (CAT) and β-galactosidase (e.g., bacterial lacZ gene) reporter systems, the firefly luciferase gene (see, e.g., Cara et al. (1996) J. Biol. Chem., 271: 5393-5397), the green fluorescence protein (see, e.g., Chalfie et al. (1994) Science 263:802) and many others. Examples of reporter plasmids are also described in U.S. Pat. No. 5,071,773. Selectable markers which facilitate cloning of the vectors of the invention are optionally included. Sambrook and Ausubel, both supra, provide an overview of selectable markers.

Typically, the reporter gene is introduced into a suitable host cell. Standard transfection methods can be used to introduce the vectors into the host cells. For mammalian host cells, preferred transfection methods include, for example, calcium phosphate precipitation (Chen and Okayama (1988) BioTechniques 6: 632), DEAE-dextran, and cationic lipid-mediated transfection (e.g., Lipofectin) (see, e.g., Ausubel, supra.).

The cells are placed in, for example, wells of a microtiter plate and allowed to become associated with a surface of the well, generally the bottom surface. The layer-forming material is added to the wells to form a barrier, with the surface-associated cells and reagents that are associated with the cells on one side of the barrier and other reagents that are not associated with the cells and which could otherwise interfere with the measurement of the reporter gene expression level (e.g., by causing background fluorescence or luminescence) on the other side of the barrier. The level of expression of the reporter gene is then assessed by measuring the amount of detectable label on either or both sides of the barrier.

In some embodiments, these methods are useful, for example, for screening to identify compounds that are suitable for use as modulators of gene expression. To conduct these methods, a regulatory region of a gene of interest (e.g., a promoter, enhancer, response element, and the like) is typically linked to a reporter gene. In some embodiments, the cells are also contacted with a potential modulator compound, before, during, or after becoming associated with the surface of the well. Cells that contain a reporter gene construct can be grown in the presence and absence of putative modulatory compounds and the levels of reporter gene expression observed in each treatment compared.

The invention also provides methods in which a layer-forming material is used to form a physical boundary between two or more populations of cells. The populations can be the same or different cell types. These methods are useful, for example, to detect diffusible effectors of cell function. In one embodiment, a first cell type that responds to a particular effector by producing a detectable signal is allowed to become associated with the bottom of the reaction vessel. The layer-forming material is then added to form a barrier, after which a second population of cells is added to the reaction vessel. If cells of the second population produce the effector of interest, and the effector can diffuse through the barrier, the surface-associated cells will produce the signal. The presence of the barrier allows one to measure the signal without need for first removing the second cell population or other components of the assay mixture.

Also provided are methods for separating and individually detecting components derived by secretion from cells, or released from cells by cell lysis. Such methods are useful, for example, for assaying cytotoxicity. The layer-forming material can also be used to form a barrier to prevent removal of cells during wash steps.

3. Compositions, Kits and Integrated Systems

The invention provides compositions, kits and integrated systems for practicing the assays described herein. For example, the invention provides a kits and assay systems that include a layer-forming material and one or more additional reagents that are useful in the assays, such as, for example, a detectable label, and a binding moiety that can specifically bind to the desired target. Also provided are assay systems and kits for cell-based screening using the methods of the invention. Such systems and kits typically include a vector that includes an appropriate reporter gene, and a suitable host cell is provided by the present invention. The kits can include any of the compositions noted above, and optionally further include additional components such as written instructions to practice a high-throughput method of conducting an assay of the invention, one or more containers or compartments (e.g., to hold reagents, nucleic acids, or the like), and an appropriate control.

The invention also provides integrated systems for high-throughput screening using the methods of the invention. The systems typically include a robotic armature which transfers fluid from a source to a destination, a controller which controls the robotic armature, a label detector, a data storage unit which records label detection, and a reaction vessel such as a microtiter plate. The integrated systems can also include, for example, a magnetic apparatus for removing a magnetic barrier layer from the wells.

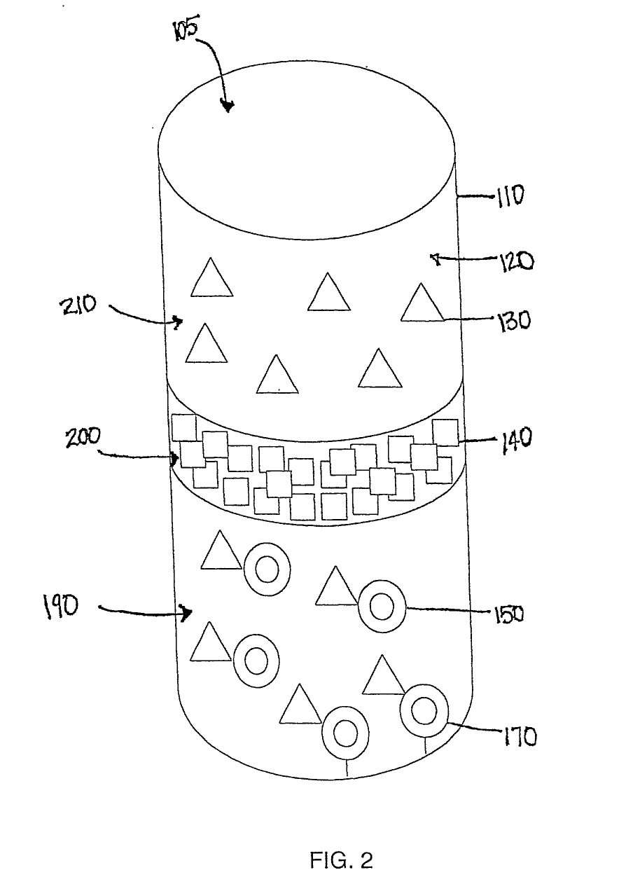

Referring now to FIG. 2, an assay measurement arrangement 100 is shown. The assay arrangement 100 generally comprises a sample 105 in an assay well 110. In the shown example, the assay well 110 contains analyte, consisting of free analyte 130 and bound analyte 150. The bound analyte 150 may be attached to a wall of the assay well 110, as illustrated by bound analyte 170. By gravity or centrifugation, the bound analyte 150 resides at the bottom of the assay well 110. The free analyte 130 floats free in sample solution 120 in the remainder of assay well 110.

Opaque material 140 is added and, for example, through gravity or low gravity centrifugation, forms layer 200 of sufficient thickness to prevent transmission of radiant energy. The layer 200 is positioned between, for example, free analyte 130 and bound analyte 150 or 170 thereby creating separate regions 190 and 210, with the layer restricting the passage of radiant energy between the separate regions 190 and 210. Thus, region 210 consists of free analyte 130 that is isolated from region 190 that consists of bound analyte 150 or 170 and is effectively separated by layer 200. The analyte in either or both regions 190 and 210 are directly and independently measurable by a detection device.

In another example, the opaque material 140 is particulate and paramagnetic. It will be appreciated that following measurement by a detection device, the opaque material 140 may be removed from the assay well 110 by applying a magnetic field that attracts the paramagnetic material from the assay well. Preferably, a magnetized pin is placed into the assay well, thereby collecting the paramagnetic opaque material. For example, a corresponding assay of magnetization pins may be placed in a corresponding array of assay wells, such as a microtiter plate, to collect the paramagnetic opaque material. However, it will be appreciated that other magnetization techniques may be used to remove the paramagnetic opaque material from the assay well.

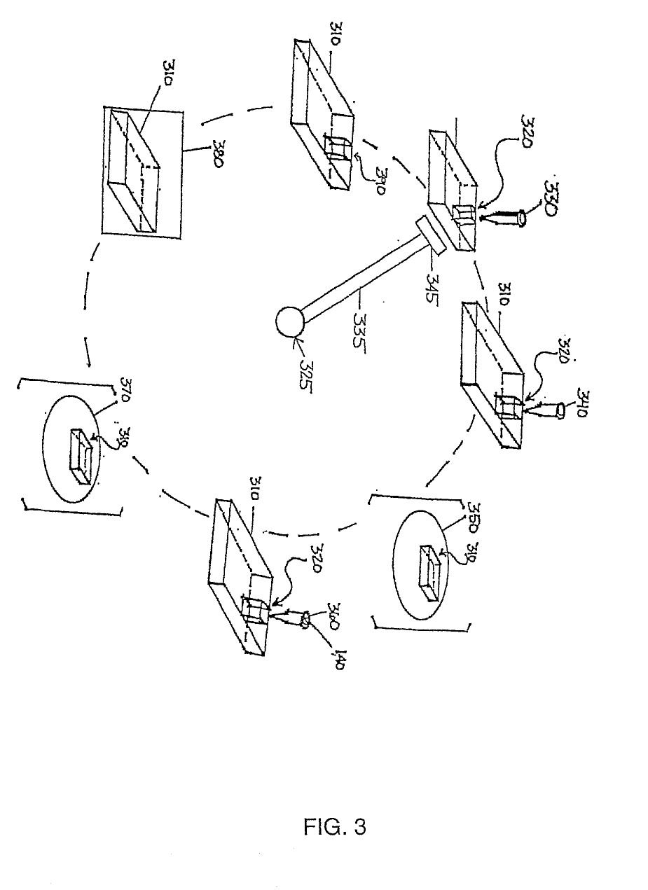

Referring now to FIG. 3, an apparatus 300 for performing an assay in accordance with the present invention is shown. Apparatus 300 comprises 310, which is positional by a robot 325. The robot 325 has a robotic arm 335 with a gripper 345 for coupling to the microtiter plate. However, it will be understood that the assay may be performed manually, robotically, or as part of a high throughput or automated system. In the shown example, a microtiter plate 310 contains an array of individual assay wells 320 (one well is illustrated). A sample, consisting for example, of analyte and reagent, is contained within assay well 320. Optionally, plate 310 may be an individual assay well or a combination of individual assay wells. It will be appreciated that other configurations of assay wells may be used.

The microtiter plate 310 is moved to the analyte dispenser 330 by the robot 325. The analyte dispenser 330 dispenses analyte into each individual assay well 320. Analyte dispenser 330 may deliver analyte to each assay well 320 sequentially, or an array of analyte dispensers may deliver analyte to an array of assay wells simultaneously. It will be appreciated that the analyte may be dispensed as a liquid, a liquid medium, or as a suspension. It will further be appreciated that dispenser 330 may be operated manually, robotically, or as part of a high throughput or automated system.

The microtiter plate 310 is moved to the reagent dispenser 340 by the robot 325. Reagent dispenser 340 dispenses reagent into each individual assay well 320. It will be appreciated that there may be more than one type of reagent being dispensed in each assay. Preferably, each reagent dispenser will dispense one type of reagent. For example, in one embodiment, the analyte consists of antigen-expressing cells. A reagent dispenser 340 dispenses an aliquot of antibody fluorescent dye conjugant into each assay well 320. It will be appreciated the reagent may be of any type suitable for an assay system, and any number of reagents may be used within an assay. It will further be appreciated that the reagent dispenser 340 may deliver reagent to each assay well 320 sequentially, or an array of reagent dispensers may deliver reagent to an array of assay wells simultaneously. It will also be appreciated that the reagent may be dispensed as a liquid, a solid, or as a suspension. Dispenser 340 may be operated manually, robotically, or as part of a high throughput or automated system.

In another embodiment, the analyte and reagent have interacted to form bound analyte and free analyte. Optionally, the bound and free analyte may be separated by employing a centrifuge 350. It will also be appreciated that the bound and free analyte may also be separated by gravity.

The plate 310 is moved to dispenser 360 by the robot 325. Opaque material 140 is added to the sample via dispenser 360 into assay well 320. It will be appreciated that the opaque material dispenser 360 may deliver opaque material to each assay well 320 sequentially, or an array of opaque material dispensers may deliver the material to an array of assay wells simultaneously. It will also be appreciated that the opaque material may be dispensed as a liquid, a solid, or as a suspension. Dispenser 360 may be operated manually robotically, or as part of a high throughput or automated system.

Optionally, a centrifuge 370 may be utilized to form the opaque material 140 into a layer 200, positioned between the bound and the free portion in the assay well, thereby creating two independent measuring regions that may be directly measured. It will be appreciated that gravity may also form the layer 200.

The plate 310 is moved to measuring device 380 by the robot 325. Using measuring device 380, the amount of analyte in region 190 and 210 is measured independently. The sample in either regions is not removed from the assay well during measurement. In preferred embodiments, measuring device 380 is a spectrophotometer, an optical system, a photomultiplier tube, or a light detector. It will be appreciated that other detection devices used in assays may be employed. Once measurements have been performed on the sample in the sample well, the assay well is removed from the measuring device, either manually or by an automated process.

It will be appreciated that assayed samples 390 may proceed to another assay system if desired. As an example, in one embodiment, the opaque material is paramagnetic. Once the samples have been measured, the opaque layer may be removed from assayed samples 390 and proceed to another sample manipulation.

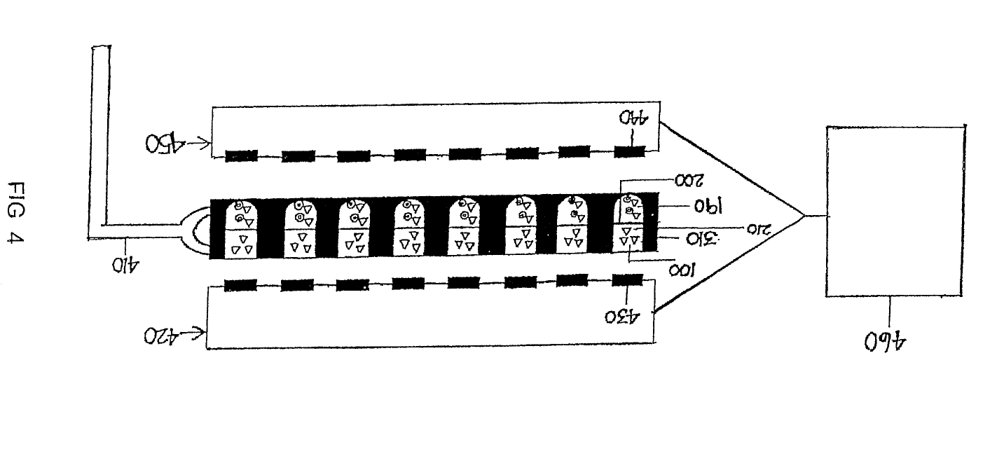

Referring to FIG. 4, a cross-sectional view of a detection device used in conjunction with a preferred embodiment of the present invention is shown. A plate handler 410 holds a sample between sensor arrays 420 and 450 of the detection device. In one embodiment, the plate handler 410 holds a microtiter plate 310, consisting of an array of assay wells, within the detection device. It will be appreciated that the plate holder may be a manual or robotic device. It will further be appreciated that the plate holder is compatible with an automated process such as a high throughput system. The radiant energy in the top region 210 of assay well 100 is detected by sensor 430, located directly above assay well 100. In a preferred embodiment, a multitude of sensors 430 is arranged in an array of sensors 420. Top sensor array 420 is located directly and correspondingly above an array of assay wells, for example, a microtiter plate, at a distance sufficient to detect the radiant energy located in the top region 210 of the assay wells. Each individual sensor 430 detects the radiant energy in the top region 210 of a corresponding assay well 100. Because of the formed layer 200, sensors 430 and 420 do not detect a statistically significant amount of radiant energy contained in bottom region 190.

Similarly, the radiant energy in the bottom region 190 of assay well 100 is detected by sensor 440, located directly below assay well 100. In a preferred embodiment, a multitude of sensors 440 is arranged in an array of sensors 450. Bottom sensor array 450 is located directly and correspondingly above an array of assay wells, for example, a microtiter plate, at a distance sufficient to detect the radiant energy located in the bottom portion 190 of the assay wells. Each individual sensor 440 detects the radiant energy in the bottom region 190 of the corresponding assay well 100. It will be appreciated that measurements made from the top sensors and the bottom sensors may be performed sequentially or simultaneously. It will further be appreciated that radiant energy may be measured in only one of the regions, if desired. Because of the formed layer 200, sensors 440 and 450 do not detect a statistically significant amount of radiant energy contained in top region 210.

Controller unit 460 coordinates the detection sensors. In a preferred embodiment, controller unit 460 is a computer. The computer coordinates data collection from the top and bottom sensors. Preferably, controller unit 460 is electronically connected to the detection device. In another preferred embodiment, the controller unit displays the data collected from the sensors. In still another embodiment, the top sensors and bottom sensors are connected to two separate controller units.

### EXAMPLES

The following examples are offered to illustrate, but not to limit the present invention.

### Example 1

A sample of Jurkat cells expressing the cell-surface antigen CD45 were labeled using a biotin-conjugated anti-CD45 antibody (Pharmigen) and placed in an assay well. Twenty μl of antibody was incubated at 4° C. for 30 minutes with 10⁶ cells in media. Unbound antibody was removed by washing cells with PBS containing 0.5% FBS and 0.1% NaN₃. A control reaction lacking antibody was processed similarly. Fifty μl of 1 μM streptavidin labeled with Q630 fluorescent dye (Quantum Dot Corporation) was added to each, incubated and washed as above. A dilution series of 250,000 to 16 cells in 50 μl total volume of PBS containing 0.5% FBS and 0.1% NaN₃ of labeled, control, and Jurkat cells were plated in a 384-well clear-bottom black microtiter plate (Greiner), consisting of an array of assay wells. Cells were pelleted to the bottom of the well by brief centrifugation for 2 minutes at 1300 rpm.

Total fluorescent label was measured using an LJL Acquest configured as follows: continuous UV lamp excitation, 450 short-pass excitation filter (Omega Optical), 630 nm FWHM35 emission filter (LJL Biosystems). Separate readings were obtained using top or bottom optics both before and after addition of the opaque material.

An opaque material was prepared by washing paramagnetic silica particles (Promega) with several volumes of water to remove the storage buffer. The opaque material was selected to restrict the passage of fluorescent energy. An equal volume of 1% BSA in TBS was added to the washed particles and incubated for approximately 1 hour. The BSA/TBS solution was removed and the paramagnetic particles were washed with several volumes of water. Particles were resuspended in a volume of water equivalent to the original volume of paramagnetic particles used. The opaque material was dispensed as a suspension.

After measuring fluorescent label as above, 20 μl of the suspension of the opaque material described above was added to each well containing cells and allowed to settle by gravity (approximately 10-20 minutes). Measurements of fluorescent label were performed using top or bottom optics as described above.

Fluorescence of Jurkat, Jurkat+fluorescent label, and Jurkat+anti-CD45+fluorescent label was determined for each sample in each assay well. Intrinsic fluorescence of the cells and background fluorescence from the plate and media were subtracted from the control (no antibody) and experimental samples.

The results show similar detection limits of cell number with or without opaque material using the bottom optics indicating that the opaque material does not interfere with fluorescence detection. The formed layer 200 is very effective in preventing light transmission from the bottom as seen by the dramatic reduction in fluorescence using the top optics. This experiment demonstrates the feasibility of using an opaque material to optically separate the top and bottom of the assay well. In this way, the relative contributions of the cells and the liquid components to the fluorescence are determined. This provides a measurement of the amount of fluorescent label bound to the cell. Essentially all of the fluorescent label was shown to be associated with the cells. This was expected since free fluorescent label was removed by washing prior to the measurement.

### Example 2

A sample of Jurkat cells expressing the cell-surface antigen CD45 were labeled using a biotin-conjugated anti-CD45 antibody (Pharmingen) and placed in an assay well. Twenty μl of antibody was incubated at 4° C. for 30 minutes with 10⁶ cells in media. Unbound antibody was removed by washing cells with PBS containing 0.5% FBS and 01.% NaN₃. Twenty μl of 1 μM streptavidin labeled with Q630 fluorescent dye (Quantum Dot Corporation) was added, incubated and washed as above. Twenty μl of biotin treated streptavidin Q580 fluorescent dye (Quantum Dot Corporation) was added to a duplicate sample to provide an unbound fluorescent label. Pre-treatment of the streptavidin Q580 conjugate with biotin prevents its association with the antibody and provides an antibody control dye. Approximately 2×10⁵ labeled cells in 40 μl total volume of PBS containing 0.5% FBS and 0.1% NaN₃ were plated in a 384 well clear-bottom black plate (Greiner). Control (unlabeled) Jurkat cells were added to adjacent wells. Cells were pelleted to the bottom of the well by brief centrifugation for 5 minutes at 1300 rpm.

Total fluorescent label was measured using an LJL Acquest configured as follows: continuous UV lamp excitation, 450 short-pass excitation filter (Omega Optical), 630 nm FWHM35 or 585 nm FWHM10 emission filter (LJL Biosystems). Separate readings were obtained using top or bottom optics both before and after addition of the opaque material.

The opaque material was prepared by washing paramagnetic silica particles (Promega) with several volumes of water to remove the storage buffer. An equal volume of 1% BSA in TBS was added to the washed particles and incubated for approximately 1 hour. The BSA/TBS solution was removed and the paramagnetic particles were washed with several volumes of water. Particles were resuspended in a volume of water equivalent to the original volume of paramagnetic particles used. The opaque material was added as a suspension.

After measuring fluorescent label as above, 20 μl of the opaque material described above was added to each well containing cells and allowed to settle by gravity (approximately 10-20 minutes). Measurements of fluorescent label were performing using top or bottom optics as described above. Background fluorescence from unlabeled control Jurkat cells was subtracted from the data.

After addition of the opaque material, signal from the Q580 label is seen exclusively using the top optics indicating that it is not associated with the cells. This is expected as binding to the antibody is prevented by pre-treatment of the Q580 label with biotin. The Q630 label is associated with the cells and is seen using the bottom optics. Some florescence is seen in the 630 nm channel using the top optics. This could be due to some dissociation of the Q630 label from the cell, some labeled cells being displaced from the bottom, or more likely due to fluorescence bleed-over from the Q580 label in the 630 nm channel. The top and bottom optics utilized different dichroic mirrors, and therefore, absolute numbers for fluorescence between top and bottom optical reads should not be compared. However, the ratios of 585 nm and 630 nm reads are valid in this case for the top and bottom optics.

### Example 3

In this example, the opaque layer is used in an FLISA format as an alternative to washing wells to remove unbound ligand. A sample of anti-luciferase antibody (Promega) was labeled with Oregon Green 488 (Molecular Probes) fluorescent dye as described by the manufacturer in protocol MP06153.

A dilution series of luciferase protein (Promega) was applied in 9 rows to a clear-bottom black Greiner 384-well microtiter plate in 0.1M NaCO₃ pH 8.3 and incubated overnight at 4° C. to bind the protein to the bottom of the well. Non-specific binding was blocked by the addition of 50 μl of 1% BSA in TBS to each well for 1 hour at room temperature. The BSA was removed and replaced with a 1:1000 dilution of the fluorescent-labeled anti-luciferase antibody prepared as above and incubated at room temperature for one hour.

The opaque material was prepared by washing paramagnetic silica particles (Promega) with several volumes of water to remove the storage buffer. An equal volume of 1% BSA in TBS was added to the washed particles and incubated for approximately 1 hour. The BSA/TBS solution was removed and the paramagnetic particles were washed with several volumes of water. Particles were resuspended in a volume of water equivalent to the original volume of paramagnetic particles used. The opaque material was dispensed as a suspension.

Total fluorescent label was measured using an LJL Acquest configured as follows: continuous UV lamp excitation, 485 nm excitation filter (LJL Biosystems), 530 nm emission filter (LJL Biosystems), bottom optics.

Triplicate rows were then treated as follows:

Rows A-C: addition of 20 μl/well of opaque material.

Rows D-F: removal of antibody, wash 3 times with TBST, add 50 μl TBST.

Fluorescent label was measured again as described above and represents the bound fluorescent label. Background fluorescence from the plate was subtracted as the average of the blank wells.

Rows D-F represent the conventional approach to performing an FLISA. Unbound antibody is removed by washing and residual fluorescence is a measure of the bound antibody. In this experiment, the assay shows a linear response from 0-81 ng of luciferase protein. Beyond this point the assay plateaus indicating a saturation of antigen binding.

Rows A-C show a response equivalent to the conventional FLISA using the opaque material. A linear response from 0-81 ng was observed and within error was superimposable with the conventional FLISA in that range. Also, the results demonstrate the assay's ability to measure analyte without additional microtiter plate washing.

### Example 4

In another example, the opaque layer is used in a genetic reporter assay format. 3T3 cells and 3T3 cells co-expressing GFP and luciferase were used at 500,000 cells/mil in DMEM media. 50, 10, or 2 μl of cells were added in triplicate to individual wells in a Greiner 384 clear-bottom microtiter plate. Total volume was adjusted to 50 μl with media. Cells were pelleted to the bottom of the well by brief centrifugation for 2 minutes at 1300 rpm.

Expression of GFP was measured using an LJL Acquest configured as follows: continuous UV lamp excitation, 485 excitation filter (LJL Biosystems), 530 nm emission filter (LJL Biosystems). Separate readings were obtained using top or bottom optics both before and after addition of the opaque material.

Luminescent measurements were performed using an LJL Acquest configured as follows: Lamp emission was blocked through a solid excitation filter, and a 600 nm short-pass emission filter (Omega optical) was used with bottom optics. 1 second integration times were used. Luciferase assay reagent (Promega) was added at 20 μl/well.

The opaque material was prepared by washing paramagnetic silica particles (Promega) with several volumes of water to remove the storage buffer. An equal volume of 1% BSA in TBS was added to the washed particles and incubated for approximately 1 hour. The BSA/TBS solution was removed and the paramagnetic particles were washed with several volumes of water. Particles were resuspended in a volume of water equivalent to the original volume of paramagnetic particles used. The opaque material was dispensed as a suspension. Twenty μl of opaque material was added and allowed to settle for approximately 10 minutes.

The results show detection of a few thousand cells expressing both GFP and luciferase reporters. Detection of GFP is possible even in the presence of media which typically interferes with low level detection of fluorescence. Luminescent detection shows similar results to GFP. The experiment demonstrates the feasibility of measuring common genetic reporters using the opaque material.

Thus, a cost effective, accurate assay measurement system that is readily automated has been provided. This assay system separates a sample into directly and independently measurable regions.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

I claim:

1. A method of performing an assay, the method comprising:
providing a sample that comprises a detectable label, wherein the sample is present in an assay vessel and at least a portion of the detectable label becomes associated with a surface of assay vessel;
adding to the sample a layer-forming material that forms a barrier between detectable label that is associated with the surface of the vessel and, if present, detectable label that is not associated the surface of the vessel; and
determining the amount of detectable label that is associated with the surface or that is not associated with the surface.

2. The method of claim 1, wherein the method comprises determining both surface-associated and surface-unassociated detectable label.

3. The method of claim 1, wherein the layer-forming material is opaque to radiant energy.

4. The method of claim 3, wherein the detectable label comprises a radiolabel and the amount of detectable label determined by adding to the sample a liquid scintillent and measuring the luminescence resulting from the interaction of the radiolabel and the scintillent.

5. The method of claim 3, wherein the detectable label emits a luminescent or fluorescent signal.

6. The method of claim 5, wherein the label comprises a luciferase, a fluorescent protein, an enzyme, a fluorescent dye, a semiconductor nanocrystal, or a calorimetric label.

7. The method of claim 1, wherein the detectable label becomes associated with the surface by binding to an analyte that binds to the surface.

8. The method of claim 7, wherein the analyte binds to the surface through a binding moiety that is attached to the surface.

9. The method of claim 7, wherein the detectable label is covalently attached to the analyte.

10. The method of claim 7, wherein the detectable label is indirectly attached to the analyte.

11. The method of claim 10, wherein the detectable label is attached to a labeling moiety that binds to the analyte.

12. The method of claim 11, wherein the labeling moiety comprises an antibody.

13. The method of claim 1, wherein the detectable label is present in a cell that becomes attached to the surface of the reaction vessel.

14. The method of claim 13, wherein the detectable label is present in periplasm or cytoplasm of the cell.

15. The method of claim 13, wherein the detectable label is present on a surface of the cell.

16. The method of claim 1, wherein the layer-forming material forms a barrier by settling onto the bottom surface of the reaction vessel.

17. The method of claim 16, wherein the settling occurs by gravity or by centrifugation.

18. The method of claim 1, wherein the layer-forming material is a paramagnetic particle.

19. The method of claim 18, wherein the method further comprises removing the paramagnetic material using a magnetic field.

20. An apparatus for performing an assay on a sample in an assay well, comprising:
means for adding to the sample a layer-forming material, thereby separating the sample into a first region and a second region; and
means for measuring a parameter of either or both of the first and second region.

21. The apparatus according to claim 20, further comprising means for forming the layer-forming material into a layer.

22. The apparatus according to claim 20, further comprising a means for measuring a parameter of the first region separately from a parameter of the second region.

23. The apparatus according to claim 22, wherein the means for measuring the parameter of the first region substantially concurrently with measuring the parameter of the second region.

24. The apparatus according to claim 22, further including means for calculating a relationship between the parameter measured from the first region and the parameter measured from the second region.

25. The apparatus according to claim 20, wherein the layer-forming material is an opaque material that restricts the passage of radiant energy from one of the first and second regions to the other.
